(19) United States
(12) Patent Application Publication
(10) Pub. No.: US 2001/0051662 A1
(43) Pub. Date: Dec. 13, 2001

(54) System and method for preparing a synthesis gas stream and converting hydrocarbons

(76) Inventors: Kym B. Arcuri, Tulsa, OK (US); Gary L. Beer, Ponca City, OK (US)

Correspondence Address: Robert H. Johnston III, Griggs & Johnston, L.L.P., North Dallas Bank Tower, Suite 1200, 12900 Preston Road, LB-15, Dallas, TX, 75230, US

(21) Appl. No.: 09/782,102

(22) Filed: Feb. 12, 2001

Related U.S. Application Data
(60) Provisional application No. 60/182,716, filed on Feb. 15, 2000.

(51) Int. Cl. C07C 27/06

(52) U.S. Cl. 518/704

(57) ABSTRACT

A system and method of operating a pre-reformer in a gas-to-liquids plant at minimum steam/carbon levels in order to reduce the C2+ content is provided that allows for the efficient production of synthesis gas to be used in producing liquid hydrocarbons preferably through a Fischer-Tropsch synthesis. The process and system involves providing steam, natural gas having greater than 3 percent C2+, and possibly hydrogen to a pre-reformer and converting a significant amount of the C2+ to methane and CO2 and then using a synthesis gas generator to prepare a synthesis gas feedstock with about a 2:1 H2:CO ratio and without undue soot formation. The synthesis gas is used in a Fischer-Tropsch synthesis unit to convert the synthesis gas to the heavier hydrocarbons. Water, light hydrocarbons, and portions of the tail gas may be recycled in different embodiments.

### BACKGROUND OF THE INVENTION

As concerns over pollution caused by traditional fossil fuels increases and as sources of crude oil decrease, there has been increased interest in other sources of energy. One promising source of energy is the synthetic production of fuels, lubricants, and other products from natural gas (referred to at times as gas-to-liquids or GTL) preferably through the Fischer-Tropsch process. See for example U.S. Pat. Nos. 4,883,170 and 4,973,453, which are incorporated by reference herein for all purposes.

A. INTRODUCTION TO THE FISCHER TROPSCH PROCESS

The synthetic production of hydrocarbons by the catalytic reaction of synthesis gas is well known and is generally referred to as the Fischer-Tropsch reaction. The Fischer-Tropsch process was developed in early part of the 20^{th} century in Germany. It has been practiced commercially in Germany during World War II and later in South Africa. See generally, www.fischer-tropsch.org.

The Fischer-Tropsch reaction for converting synthesis gas (primarily CO and H₂) has been characterized by the following general reaction:

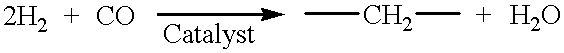

The hydrocarbon products derived from the Fischer-Tropsch reaction range from methane to high molecular weight paraffinic waxes containing more than 100 carbon atoms.

Numerous Fischer-Tropsch catalysts (e.g., Group VIII metals on various supports such as alumina and titania and with various promoters) have been used in carrying out the reaction, and both saturated and unsaturated hydrocarbons can be produced. The synthesis reaction is very exothermic and temperature sensitive whereby temperature control is required to maintain a desired hydrocarbon product selectivity.

B. INTRODUCTION TO SYNTHESIS GAS PRODUCTION

Synthesis gas may be made from natural gas, gasified coal, and other sources. A number of methods have been employed for producing the synthesis gas (“syngas”), which is substantially carbon monoxide and molecular hydrogen. Synthesis gas is utilized as feedstock in the Fischer-Tropsch reaction. The numerous methodologies and systems that have been used to prepare synthesis gas include partial oxidation, steam reforming, auto-reforming or autothermal reforming, and secondary reforming. Both fixed and fluid bed reactor systems have been employed.

The reforming reactions are endothermic, and a catalyst containing nickel is often utilized. Partial oxidation (catalytic or non-catalytic) involves sub-stoichiometric combustion of light hydrocarbons such as methane to produce the synthesis gas. The partial oxidation reaction is typically carried out commercially using high purity oxygen.

In some situations these synthesis gas production methods may be combined to form another method. A combination of essentially partial oxidation and steam reforming, known as autothermal reforming, wherein air (or O₂) is used as a source of oxygen for the partial oxidation reaction has also been used for producing synthesis gas heretofore. Autothermal and secondary reforming are a combination of partial oxidation and steam reforming where the exothermic heat of the partial oxidation supplies the necessary heat for the endothermic steam reforming reaction. The autothermal reforming process can be carried out in a relatively inexpensive refractory lined carbon steel vessel whereby low cost is typically involved.

The autothermal process results in a lower hydrogen to carbon monoxide ratio in the synthesis gas than does steam reforming alone. That is, the steam reforming reaction with methane results in a ratio of about 3:1 or higher while the partial oxidation of methane results in a ratio of less than about 2:1—depending upon the extent of the water gas shift reaction. A good ratio for the Fischer-Tropsch hydrocarbon synthesis reaction carried out at low or medium pressure (i.e., in the range of about atmospheric to 500 psig (3447.4 KPa) over a cobalt catalyst is about 2:1. When the feed to the autothermal reforming process is a mixture of light shorter-chain hydrocarbons, such as a natural gas stream, some form of additional control is desired to maintain the ratio of hydrogen to carbon monoxide in the synthesis gas at the optimum ratio (for cobalt based FT catalysts) of about 2:1. For this reason, steam and/or CO₂ may be added to the synthesis gas reactor to adjust the H₂/CO ratio to the desired value with the goal of optimizing process economics.

Two-zone synthesis gas systems are generally known. One known approach is to begin with an oxidation step in which most, if not all, of the molecular oxygen is consumed. The heat release from the oxidation reactions raises the gas temperature to values typically beyond 2500 F. (1371 C.). In many cases the oxidation reactions are carried out in an open volume free of any catalyst. This flame or combustion zone is designed to withstand very high temperatures (>2800 F. or >1538 C.). The hot gases then pass through a bed of reforming catalyst where the endothermic reaction between the unoxidized hydrocarbons and water proceeds to produce CO and H2 at the desired ratio. The water gas shift reaction proceeds to equilibrium as well as the reforming reaction and should be accounted for in order to achieve the desired H2/CO ratio for Fischer-Tropsch synthesis (approximately 2/1).

As previously noted, the preferred H2/CO ratio in many GTL processes is typically around 2.0. Much of the existing literature, however, appears to deal with producing synthesis gas at higher ratios (e.g., 3). Most commercial applications involving synthesis gas production are for hydrogen and/or methanol production. In methanol applications, the desired H2/CO ratio in the synthesis gas is on the order of 2.5 or higher. In hydrogen production, the exit ratios of H2/CO are much higher. Other applications such as those involving hydrogen cyanide production typically involve ratios greater than 2.5/1.

Steam may be used to control the H2/CO ratio. Increasing the amount of steam in the feed gas results in higher H2/CO ratios via the water gas shift and hydrocarbon reforming equilibrium. Conversely, lowering the steam level leads to lower H2/CO ratios. Consequently when relatively low H2/CO ratios are desired (i.e. 2/1 or lower) it is necessary to operate with a low amount of steam in the NG feed.

One problem in preparing synthesis gas to which considerable attention has been focused is avoiding soot. Soot and carbon formation is an unwanted side result in synthesis gas generation. This unwanted product can be produced by thermal pyrolysis of hydrocarbons and/or disporportination of the CO product. It is a fact that hydrocarbons can undergo thermal pyrolysis to produce gas phase carbon (or soot) and that they can readily crack to carbon on a hot catalytic surface. The presence of steam mitigates these unwanted reactions. When producing synthesis gas at ratios greater than 2/1 H2/CO, soot or carbon formation is minimized due to the higher water levels in the feed. When employing natural gas mixtures containing significant amounts of C2+ with the intent on generating synthesis gas with a H2/CO ratio of about 2.0, a minimum amount of water is added to maintain the 2:1 ratio. When producing synthesis gas mixtures at approximately a 2/1 H2/CO one must employ small amounts of steam. The tendency to produce soot or carbon, however, greatly increases, especially when utilizing natural gas resources containing significant amounts of C2+ components (e.g., >3%).

### SUMMARY OF THE INVENTION

Therefore, a need has arisen for a system and method for effectively preparing a synthesis gas stream and converting hydrocarbons that addresses shortcoming of the previous systems and methods. According to an aspect of the invention, a method of operating a pre-reformer in a gas-to-liquids plant at minimum steam/carbon levels in order to reduce the C2+ content is provided that allows for the efficient production of synthesis gas to be used in producing liquid hydrocarbons preferably through a Fischer-Tropsch synthesis.

According to an aspect of the present invention, a process for converting light hydrocarbons to heavier hydrocarbons (C5+) includes the steps of providing steam, natural gas having greater than 3 percent C2+, and hydrogen to a pre-reformer; using the pre-reformer to convert a significant amount of the C2+ to methane and CO2 (or alternatively to just remove C2+) to thereby prepare a synthesis gas feedstock; delivering the synthesis gas feed stock to a synthesis gas generator; using the synthesis gas generator to produce a synthesis gas having about a 2:1 H2:CO ratio without significant soot or carbon formation in the synthesis gas generator; and delivering the synthesis gas to a Fischer-Tropsch synthesis unit; and using the Fischer-Tropsch synthesis unit to convert the synthesis gas to the heavier hydrocarbons.

According to another aspect of the present invention, a pre-reformer that receives natural gas with more than 3% C2+ is used as part of a hydrocarbon conversion system to convert a significant amount of the C2+ in the synthesis gas feed stock to methane and CO2, CO and H2 (but mainly to remove C2+). Wastewater is delivered for use in the pre-reformer. According to another aspect of the present invention, a pre-reformer that receives natural gas with more than 3% C2+ is used as part of a hydrocarbon conversion system to convert a significant amount of the C2+ in the synthesis gas feed stock to methane and CO2 (and possibly CO and H) and a synthesis unit is used to prepare an FT heavy product stream, an FT light product stream, and a tail gas wherein a portion of the light FT product is returned for use in the pre-reformer. According to yet another aspect of the present invention, a scrubber may be used to remove CO2 from the tail gas and the CO2 returned to the pre-reformer for use therein.

According to another aspect of the present invention, a pre-reformer that receives natural gas with more than 3% C2+ is used as part of a hydrocarbon conversion system or process to convert a significant amount of the C2+ in the synthesis gas feed stock to methane and CO2 (and other items without C2+) and a synthesis unit is used to prepare an FT heavy product stream, an FT light product stream, and a tail gas and wherein the heavy FT product stream is delivered to a hydrocarbon upgrading unit and wherein any light hydrocarbons generated in the hydrocarbon upgrading unit are delivered to the pre-reformer for use therein

The present invention provides many advantages. For example, an advantage of the present invention is that one can employ natural gas with significant amounts (e.g., >3%) of C2+ when generating synthesis gas with an H2/CO ratio of approximately 2.0 without undue soot or carbon formation. This is especially beneficial in remote sites with untreated natural gas containing high levels of C2+ constituents.

### BRIEF DESCRIPTION OF THE DRAWINGS

For a more complete understanding of the present invention and advantages thereof, reference is now made to the following description taken in conjunction with the accompanying drawings in which like reference numbers indicate like features, and wherein:

FIG. 1 is a schematic diagram of a plant system for generating a synthesis gas stream and converting hydrocarbons according to a first embodiment of the present invention; and

FIG. 2 is a schematic diagram of a plant system for generating a synthesis gas stream and converting hydrocarbons according to a second embodiment of the present invention.

### DETAILED DESCRIPTION OF THE INVENTION

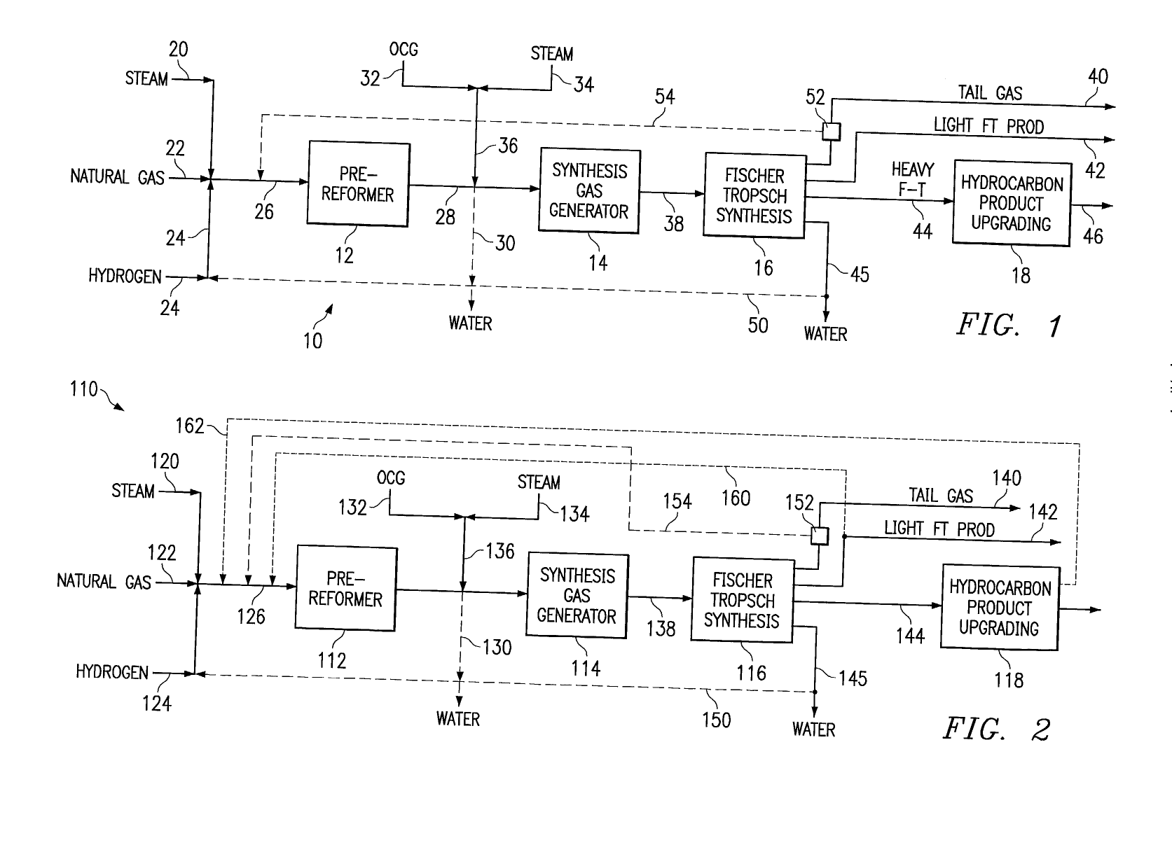

The preferred embodiment of the present invention and its advantages are best understood by referring to FIGS. 1-2 of the drawings, like numerals being used for like and corresponding parts of the various drawings.

Referring to FIG. 1, a system 10 for preparing a synthesis gas stream and for converting hydrocarbons is presented. As an important aspect of the present invention, an adiabatic pre-reformer 12 is used to prepare a feed stock (preferably a single feed stock stream) before delivery to a synthesis gas generator 14. The synthesis gas generator 14 is used to prepare synthesis gas that is used in a synthesis unit 16, which is preferably a Fischer-Tropsch synthesis unit. A portion of the products from the synthesis unit 16 may be upgraded in a hydrocarbon product-upgrading unit 18 as will be described further below.

Steam, natural gas, and hydrogen are delivered to system 10 through conduits 20, 22, and 24, respectively. The exact composition of the natural gas will vary between reservoirs with some containing low or high concentrations of various compounds including nitrogen, water vapor, carbon dioxide, helium, hydrogen sulfide or ethane and heavier paraffinic hydrocarbons. The feed stocks 20, 22, 24 are mixed and delivered by conduit 26 to pre-reformer 12. The feed stocks are conditioned (e.g., liquids removed; sulphur removed from the natural gas and compressed) before delivery to the pre-reformer 12. The natural gas may go through a bulk sulphur removal, then be heated, and then a final sulphur removal may occur. The sulphur removal may result in only 1 ppmv or less sulphur in the feedstock. In pre-reformer 12, the heavier constituents are converted such that methane, CO₂, and possibly H2 and CO are prepared for delivery, but it primarily exists to remove C2+. No refrigeration or expansion type gas plant is required to reduce the quantity of paraffinic hydrocarbons heavier than methane.

The pre-reformer 12 addresses the tendency for soot and carbon to form in the synthesis gas generator 14—especially when using natural gas resources containing significant amount of C2+ (e.g., >3%) as may occur with untreated gas in remote locations. The pre-reformer 12 helps to minimize the C2+ content in the natural gas feed to the syngas generator of a GTL plant.

Pre-reformers have been utilized in other commercial applications involving the conversion of naptha to methane and the removal of trace quantities of heavier constituents from methane rich streams (referred to as “de-enriching”). It is believed that in all or the vast majority of the current pre-reforming commercial applications, the central objective is only to produce a synthesis gas with a H2/CO ratio well in excess of 2—typically 3.0. In contrast, the pre-reformer 12 of the present invention is devoted to minimizing the amount of water in the feed in order to optimize thermal efficiency in the overall process. Minimizing the water allows one to achieve H2:CO ratio of about 2:1 in the synthesis gas for use in a GTL plant. Further, it allows for undue soot and carbon formation to be avoided.

The pre-reformer 12 is an adiabatic reformer that is of a type suitable for use with hydrocarbon feed stocks having a significant amount of C2+. The pre-reforming process is preferably carried out in a fixed bed adiabatic reactor that is upstream of the primary synthesis gas generator 14. The pre-reformer 12 uses a highly-active reforming catalyst suitable for use at low temperatures; a commonly used reformer catalysts is Ni/Al₂O₃. A suitable reforming catalyst is commercially available from Synetix (United Kingdom); the catalyst contains high Nickel levels, typically greater than 40 wt. %. Other catalyst from commercial suppliers contain Ni levels on the order of about 20-40 wt. Percent.

The pre-reformer 12 converts a significant amount of the C2+ to methane through the following reactions represented by the pentane constituent:

C₅H₁₂+5H₂O=5 CO+11 H₂ (1)

5 CO+15 H₂=5 CH₄+5 H₂O (2)

CO+H₂O=CO₂+H₂ (3)

The ideal operating scenario is to have all three reactions operating at equilibrium conversion levels. The reactions can be used to merely eliminate C2+ and can be carried out to also have CO and H2. Additional hydrogen may be needed to drive the methanation reaction and the addition of steam may be necessary to increase the conversion of the heavier constituents while minimizing any soot formation.

The production of synthesis gas in GTL operations (operating at a nominal 2/1 H2/CO ratio) is most efficient when there is a minimum amount of steam present. The synthesis gas reactions associated with GTL operations are as follows. The following equations employ methane as the example of the hydrocarbon reactant. Similar equations can be used for heavier constituents.

CH₄+O₂=CO+H₂O+(in some cases) CO₂ (4)

CH₄+H₂O=CO+O2 H₂ (5)

CO+H₂O=CO₂+H₂ (6)

The presence of high levels of heavier constituents such as propane or pentane can lead to soot formation through thermal pyrolysis reactions. In the absence of sufficient oxygen for oxidation, the heavier hydrocarbons can undergo unimolecular reactions to form free radicals at the desired operating temperatures for the equilibrium conversion of methane (equation 4 above). An example of the soot formation reactions for pentane can be written as follows:

C₅H₁₂→carbon and associated hydrocarbon radicals (7)

These free radicals can combine or otherwise revert to olefins, aromatics, and soot that leads to synthesis gas generator catalyst bed plugging and other mechanical problems. Additionally, heavier hydrocarbons and their free radicals can also form soot or carbon on the catalyst surface if the presence of steam minimizes these reactions.

It is known in some applications to convert the heavier constituents to methane through the addition of steam and hydrogen. The known pre-reformers typically use steam at sufficiently high levels (>0.8 moles steam per mole carbon in hydrocarbon feed). The presence of these steam levels promotes the generation of hydrogen through the water gas shift reaction described in equation (6) above. Increasing the steam level leads to the conversion of CO to form hydrogen and CO₂. Consequently, the hydrogen to CO ratio in the equilibrium product synthesis gas for these known applications is higher than the desired 2:1 value desired for most GTL/Fischer-Tropsch operations. The 2:1 is particularly desirable for low or medium pressure synthesis over cobalt-based Fischer-Tropsch catalysts.

Low steam levels (<0.8 moles steam to moles carbon in the C2+ hydrocarbon feed constituents for low CO2 concentrations) are preferably used with the pre-reformer 12. The preferred amount of steam is the minimum amount that allows a high degree of conversion of the C2+ constituents in the pre-reformer 12. A high degree of conversion does not necessarily signify equilibrium conversion—and in many instances will not—since the primary intent is to reduce the content of the heavier constituents to minimize soot and carbon formation. Reduction to levels less than 1 vol % are sufficient especially when the residual heavier material (C3+) is essentially completely converted to nearly (>90%) the equilibrium conversion level. Note that in GTL applications, pre-reforming is not needed or considered to reduce the BTU or heat content of the hydrocarbon gas to the synthesis gas production unit; de-enriching is not a concern. Pre-reformer 12 is to minimize the amount of heavier constituents to prevent carbon formation while operating at the relatively low steam to carbon ratios necessary to achieve the desired H2/CO ratio (nominally 2).

The pre-reformer 12 in system 10 minimizes the amount of heavier hydrocarbon constituents while allowing operations at low steam/carbon ratios. The effluent (conduit 28) from the pre-reformer 12 may contain a relatively low steam/carbon mole ratio (preferably less than 0.4). This ratio is defined as the moles of steam to the total moles of carbon contained in the hydrocarbons and CO2. The C2+ content is preferably less than 1 vol % with the condition that all of the C3+ constituents have been converted in accordance to the maximum possible equilibrium conversion. The product gas from the pre-reformer 12 is then combined with an oxygen-containing gas (OCG) 32 (e.g., air, enriched air, etc.) and then passed to the synthesis gas generator 14.

The steam level in the effluent from the pre-reformer 12 may have to be adjusted to achieve the optimal composition necessary to obtain the 1.8 to 2.2 H2/CO ratio desired for GTL applications. In cases where there are relatively high concentrations of CO2 in the effluent gas from the pre-reformer 12, one may need to add additional steam 34. In applications where there are significantly high levels of C2+ in the hydrocarbon source (i.e. pre-reforming naptha), steam levels in the pre-reformer 12 may be too high to allow efficient conversion of the pre-reforming effluent to a synthesis gas with a 2/1 H2/CO ratio. Consequently, the excess steam can be removed as depicted in conduit 30. There may be limited cases in which the pre-reformer 12 produces excessive amounts of CO2 and/or the incomplete conversion of the heavier constituents require the addition of additional steam to allow efficient conversion to the desired H2/CO for FT processing.

After pre-reforming as described above, the effluent of pre-reformer 12 is delivered to conduit 28. Because the effluent of pre-reformer 12 may contain water levels that are in excess of that required to achieve the desired H₂/CO synthesis gas ratio, water may be removed as suggested by conduit 30. Steam and an oxygen-containing gas (OCG) may be introduced into conduit 28 before delivery to synthesis gas generator 14. The oxygen-containing gas, which may be oxygen or air or enriched air, for example, is shown being introduced into system 10 through conduit 32. The steam/water is shown being introduced through conduit 34. The water source to the pre-reformer 12 can be either normal utility source or recycled water from the FT reaction (i.e. water of conduit 45 may be delivered to conduit 20 or conduit 26 as suggested in the embodiment by conduit 50). Recycled FT water offers the advantage of containing additional organic materials (alcohols and other oxygenates) which are otherwise unused and treated as waste material. Recycling this material to the pre-reformer 12 offers the opportunity to convert these materials to valuable liquid hydrocarbon products (through conversion to methane in pre-reformer 12 and CO in syngas generator 14).The OCG and/or steam are delivered to conduit 28 through conduit 36.

Synthesis gas generator 14 receives its feed stock through conduit 28 and produces a synthesis gas that is delivered by conduit 38 to synthesis unit 16. Synthesis gas generator 14 may use any of a number of units for producing synthesis gas. For example, an autothermal reformer, a steam methane reformer, or partial oxidation unit. An autothermal reformer is preferred.

The synthesis gas developed in synthesis gas generator 14 is delivered by conduit 38 to synthesis unit 16, which is preferably a Fischer-Tropsch synthesis unit. Through the Fischer-Tropsch reaction, the synthesis gas is converted to heavier hydrocarbons (C5+) relative to the natural gas feed of conduit 22. The hydrocarbons can be converted from methane all the way to heavy paraffinic waxes containing more than 100 carbon atoms in an alkane chain.

The light incondensible gases forming the effluent of synthesis unit 16 are delivered to conduit 40. This effluent or tail gas may contain H₂, CO, H₂O, CO₂, and the full range of alkane hydrocarbons. The tail gas of conduit 40 may be used at a number of locations within system 10 as a fuel gas; for example, it may be used to supply burner fuel or may be used as a fuel for a turbine combustor if one is utilized as part of synthesis gas generator 14. See, e.g., U.S. Pat. Nos. 5,733,941 and 5,861,441 which are incorporated herein by reference for all purposes. Some olefins and oxygenated hydrocarbons can also be produced. When air or enriched air is introduced through conduit 32, the tail gas may also contain significant amounts of nitrogen (e.g. greater than 5 mole percent). The tail gas may also have the CO2 content of it scrubbed by scrubber 52 and the resultant CO2 stream delivered by conduit 54 to conduit 26 for use in the pre-reformer 12. The additional CO2 delivered to the pre-reformer 12 may use more H2, but that may be adjusted directly or through the addition of steam. The CO2 removal and recycle facilitates CO2 production in the pre-reformer 12 and will make the remaining tail gas downstream of scrubber 52 more combustible.

The light Fischer-Tropsch products (e.g., BP≦50 F. (BP≦10 C.)) developed in synthesis unit 16 are delivered to conduit 42 from where they may go to storage, and the heavy Fischer-Tropsch products (e.g., BP≧200 F.(BP≧93 C.)) are delivered by conduit 44 to hydrocarbon product upgrading unit 18 (or optionally to storage). The distinction between light and heavy FT products in the previous sentence can vary over a significant boiling-point ranges—50 F. (10 C.) and 200 F. (93 C.) are for illustration. The hydrocarbon product upgrading unit 18 may be used for hydrotreating and/or hydro-isomerization or otherwise to treat heavy FT product to produce other desired hydrocarbons which may then be delivered by conduit 46 to storage.

Conduit 45 carries away waste by-product water for treatment and disposal or for use elsewhere in system 10. If used elsewhere in system 10, it may be delivered directly by conduit 50 to conduit 24 for use as a steam/water source. The oxygenates in the water and any unwanted or low-value constituents in it are converted to part of the feed stock for the pre-reformer 12.

Referring now to FIG. 2, a system 110 for preparing a synthesis gas stream and converting hydrocarbons is presented. System 110 is analogous in most respects to system 10 of FIG. 1. The analogous components and sub-systems have the same reference numerals with 100 added to them. In system 110, a recycle conduit 160 returns a portion of the light hydrocarbons (typically less than 200 F. BP (93 C.). The light hydrocarbons are recovered and sent back to the inlet 126 to the pre-reformer 112. Recycle conduit 162 has also been added. Recycle conduit 162 recycles light hydrocarbons generated through the hydro-treating and hydro-isomerization of the liquid products in product upgrading unit 118.

The light (non-liquid) components in conduit 160 can be recycled since upgrading to higher valued products is generally not practical. Generally hydrocarbon constituents from C1 to the carbon number in which collection for further upgrading becomes impractical, can be recycled since conversion to CO followed by subsequent synthesis to heavier products is beneficial. Conduit 160 depicts the recycle loop by which hydrocarbons produced in the FT step can be recycled back to the feed to the pre-reformer 112. The steam and oxygen rates to the pre-reformer 112 must be suitably adjusted to allow for the effective conversion of the additional hydrocarbons brought into the natural gas stream 122 through recycle.

Product upgrading can also generate quantities of light hydrocarbons through unwanted cracking reactions associated with hydro processing. These light gases (typically C4 and lighter) can be recycled back through conduit 162 to the inlet 126 of the pre-reformer 112 and combined with the natural gas stream 122. The steam 134 and oxygen-containing gas 132 rates to the pre-reformer must be suitably adjusted to allow for the effective conversion of the additional hydrocarbons brought into the natural gas stream 122 through recycle.

Another important aspect of the invention involves effectively managing the light hydrocarbons, which can have marginal or a negative value in a GTL plant designed to produce high-valued liquid products. The use of a pre-reformer to convert the C2+ constituents into methane through a combination of reforming and methanation are applied to a GTL system. This stands in contrast to using a pre-reformer to reduce the BTU content of the feed gas (i.e., to de-enrich). The importance of reducing the C2+ content in order to allow for the production of synthesis gas with a H2/CO ratio of nominally 2 has been utilized as presented here.

The systems, and methods of the present invention are preferably used, at least in part, to convert synthesis gas into longer-chain hydrocarbons, e.g., the full spectrum of C₅₊ products through the Fischer-Tropsch reaction (but the invention further has application with other non-Fischer-Tropsch processes). There are an infinite number of different ways to split the Fischer-Tropsch products according to the specific market needs. For example if the desired hydrocarbon products are destined for a fuels market (diesel and jet fuel), the liquid products destined for product upgrading can involve relatively low carbon numbers of C₉₋₁₄. If the products involve predominantly lube oils and other high molecular weight specialty products, the desired hydrocarbon products must have carbon numbers greater than approximately C14.

In synthesis unit 16 and 116, the synthesis gas is converted to a mixture of hydrocarbons ranging from light gases (methane, ethane, propane), light naptha (C4 to C6 alkanes) and heavy “liquid” products ranging from C7 to C100 or heavier. The definition of liquid products can take on many forms depending upon the ultimate hydrocarbon products sent to market. For example, in some cases hydrocarbons heavier than C10 may be considered valuable liquids and in other cases products starting at C5 and heavier can be considered liquid products. In cases where C2+ constituents are considered valuable (feed stocks and/or NG liquid markets) all hydrocarbons heavier than methane can be considered products.

The FT products contain a range of hydrocarbons and gases including unreacted feed, carbon dioxide as well as inerts, which came in with the air and/or natural gas. In most cases the product is predominantly paraffinic; however, there can be significant amounts of olefins and oxygenated hydrocarbons including, but not limited to, carboxylic acids, alcohols, and aldehydes. A hydrocarbon upgrading unit 18 and 118 is desirable in many circumstances to convert the olefins and oxygenates to the desired paraffinic alkanes which can be used in a multitude of high-valued products including lubrication oils, waxes, synthetic diesel and jet fuels, and other fuels. The upgrading unit 18 and 118 can also include an isomerization step to increase the branching of the saturated paraffins there by increasing their value and utility in specialty lubricants, fuels and other products. The longer-chain Fischer-Tropsch products that may be made directly or with downstream processing include numerous products for numerous uses. The literature and patents shows a plethora of possible products.

Although the present invention and its advantages have been described in detail, it should be understood that various changes, substitutions and alterations can be made therein without departing from the spirit and scope of invention as defined by the appended claims. Further it should be understood, the numerous additional components and devices and steps as known in the art may be applied in carrying out the system and methods presented here; for example, there would be numerous conventional heat exchangers and separators as shown, for example, in U.S. Pat. No. 4,973,453, and as well known in the art.

What is claimed is:

1. A process for converting light hydrocarbons to heavier hydrocarbons (C5+) involving the Fischer-Tropsch reaction, the process comprising the steps of:
providing steam, natural gas having greater than 3 percent C2+, and hydrogen to a pre-reformer;
using the pre-reformer to convert more than 50 percent of the C2+ to methane and CO2 to thereby prepare a synthesis gas feedstock;
delivering the synthesis gas feed stock to a synthesis gas generator;
using the synthesis gas generator to produce a synthesis gas having about a 2:1 H2:CO ratio without significant (i.e., not hindering operation) soot or carbon formation in the synthesis gas generator;
delivering the synthesis gas to a Fischer-Tropsch synthesis unit; and
using the Fischer-Tropsch synthesis unit to convert the synthesis gas to the heavier hydrocarbons.

2. The process of claim 1 wherein the system creates by-product waste water and constituents and further comprising the step of delivering the by-product waste water and waste constituents to the pre-reformer for use therein.

3. The process of claim 1 further comprising the steps of:
using the synthesis unit to prepare a heavy FT product stream, a light FT product stream, and a tail gas; and
delivering the heavy FT product to a hydrocarbon product upgrading unit; and
using the hydrocarbon upgrading unit to modify the heavy FT product stream.

4. The process of claim 3 further comprising the steps of:
delivering the tail gas to a CO2 scrubber;
using the CO2 scrubber to remove CO2 therefrom; and
delivering the CO2 to the pre-reformer for use therein.

5. The process of claim 3 further comprising the steps of:
removing a portion of the tail gas (BP<200 F. (BP<93 C.)); and
recycling the portion of the tail gas removed to the pre-reformer for use therein.

6. The process of claim 3 further comprising the steps of removing any light hydrocarbons generated in the hydrocarbon upgrading unit and delivering the light hydrocarbons from the upgrading unit to the pre-reformer for use therein.

7. The process of claim 3 wherein the step of using the pre-reformer to convert the C2+ to methane and CO2 to thereby prepare a synthesis gas feedstock comprises: using a pre-reformer to carry out the reactions represented by the following equations:
CₙH₂ₙ₊₂+n H₂O=n CO+(2n+1)H₂ n=3, 4, 5
5 CO+15 H₂=5 CH₄+H₂O
CO+H₂O=CO₂+H₂.

8. The process of claim 7 wherein the reactions are not carried out to equilibrium.

9. A system for converting light hydrocarbons to heavier hydrocarbons (C5+) comprising:
a pre-reformer operable to receive steam, natural gas having greater than 3 percent C2+, and hydrogen and operable to convert a significant amount of C2+ to methane and to thereby prepare a synthesis gas feedstock;
a synthesis gas generator fluidly coupled to the pre-reformer for receiving a synthesis gas feedstock and operable to produce a synthesis gas have about a 2:1 H2:CO ratio without significant soot or carbon formation in the synthesis gas generator; and
a synthesis unit fluidly coupled to the synthesis gas generator for receiving synthesis gas from the synthesis gas generator and converting the synthesis gas to heavier hydrocarbons.

10. A process for converting light hydrocarbons to heavier hydrocarbons (C5+), the process comprising the steps of:
(A) providing steam, natural gas (having greater than 3 percent C2+), and hydrogen to a pre-reformer;
(B) using the pre-reformer to convert more than 50 percent of the C2+ to methane and CO2 to thereby prepare a synthesis gas feedstock, wherein this step (B) involves the following reactions:
C₅H₁₂+5 H₂O=5 CO+11 H₂
5 CO+15 H₂=5 CH₄+5 H₂O
CO+H₂O=CO₂+H₂;
(C) delivering the synthesis gas feed stock to a synthesis gas generator;
(D) using the synthesis gas generator to produce a synthesis gas having an H2:CO ratio of about 1.8:1 to 2.2:1 and wherein the synthesis gas is produced without any significant soot or carbon formation in the synthesis gas generator;
(E) delivering the synthesis gas to a Fischer-Tropsch synthesis unit;
(F) using the Fischer-Tropsch synthesis unit to convert the synthesis gas into a heavy FT product stream, a light FT product stream, and a tail gas;
(G) delivering the heavy FT product stream produced in step (G) to a hydrocarbon product upgrading unit;
(H) using the hydrocarbon upgrading unit of step (G) to modify the heavy FT product stream;
(I) delivering the tail gas produced in step (F) to a CO₂ scrubber;
(J) using the CO₂ scrubber to remove CO₂ from the tail gas;
(K) delivering CO₂ from step (J) to the pre-reformer for use in step (B);
(L) removing at least a portion of the tail gas (BP<200 F.) prepared in step (F);
(M) recycling the portion of the tail gas removed in step (L) to the pre-reformer for use in step (B); and
(N) removing any light hydrocarbons generated in the upgrading of step (H) to the pre-reformer for use in step (B).
